SEQUENCE LISTING (5 sequences)

SEQ ID NO: 1 - protein, 21 aa, Homo sapiens
GIVEQCCTSICSLYQLENYCN

SEQ ID NO: 2 - protein, 30 aa, Homo sapiens
FVNQHLCGSHLVEALYLVCGERGFFYTPKT

SEQ ID NO: 3 - protein, 30 aa, Artificial sequence
  B chain of insulin lispro
FVNQHLCGSHLVEALYLVCGERGFFYTKPT

SEQ ID NO: 4 - protein, 30 aa, Artificial sequence
  B chain of insulin aspart
FVNQHLCGSHLVEALYLVCGERGFFYTDKT

SEQ ID NO: 5 - protein, 30 aa, Artificial sequence
  B chain of insulin glulisine
FVKQHLCGSHLVEALYLVCGERGFFYTPET

(19) United States
(12) Patent Application Publication
(10) Pub. No.: US 2020/0093894 A1
(43) Pub. Date: Mar. 26, 2020

(54) NOVEL FORMULATIONS

(71) Applicant: Arecor Limited, Saffron Walden (GB)

(72) Inventors: Jan JEZEK, Saffron Walden (GB); David GERRING, Saffron Walden (GB); Sarah HOWELL, Saffron Walden (GB); Leon ZAKRZEWSKI, Saffron Walden (GB)

(21) Appl. No.: 16/610,805

(22) PCT Filed: May 3, 2018
(86) PCT No.: PCT/GB2018/051178

§ 371 Date: Nov. 4, 2019

(30) Foreign Application Priority Data
May 5, 2017 (GB) 1707187.9

(51) Int. Cl. A61K 38/28; A61K 31/455; A61K 47/26; A61K 47/10; A61K 47/02; A61K 9/00; A61K 9/08

(52) U.S. Cl. CPC A61K 38/28; A61K 31/455; A61K 47/26; A61K 9/08; A61K 47/02; A61K 9/0019; A61K 47/10

(57) ABSTRACT

The present invention relates inter alia to an aqueous liquid pharmaceutical formulation comprising (i) an insulin compound, (ii) ionic zinc, (iii) a nicotinic compound (iv) a non-ionic surfactant; and (v) a salt selected from the salts formed between Group 1 metals and a mono or divalent anion. It also provides related methods, uses and pharmaceutical compositions.

### FIELD OF THE INVENTION

This invention relates inter alia to rapid acting aqueous liquid formulations of insulin and insulin analogues. Such formulations are suitable for the treatment of subjects suffering from diabetes mellitus, especially Type 1 diabetes mellitus.

### BACKGROUND OF THE INVENTION

Diabetes mellitus (“diabetes”) is a metabolic disorder associated with poor control of blood sugar levels leading to hypo or hyperglycemia. Untreated diabetes can lead to serious microvascular and macrovascular complications including coronary artery disease, peripheral artery disease, stroke, diabetic nephropathy, neuropathy and retinopathy. The two main types of diabetes are (i) Type 1 diabetes resulting from the pancreas not producing insulin for which the usual treatment is insulin replacement therapy and (ii) Type 2 diabetes where patients either produce insufficient insulin or have insulin resistance and for which treatments include insulin sensitising agents (such as metformin or pioglitazone), traditional insulin secretagogues (such as sulfonylureas), SGLT2 inhibitors (such as dapagliflozin, canagliflozin and empagliflozin) which reduce glucose absorption in the kidneys and so promote glucose excretion, GLP-1 agonists (such as exenatide and dulaglutide) which stimulate insulin release from pancreatic beta cells and DPPIV inhibitors (such as sitagliptin or vildagliptin) which inhibit breakdown of GLP-1 leading to increased insulin secretion. Patients with Type 2 diabetes may eventually require insulin replacement therapy.

For patients requiring insulin replacement therapy, a range of therapeutic options are possible. The use of recombinant human insulin has in recent times been overtaken by use of insulin analogues which have modified properties, for example, are longer acting or faster acting than normal insulin. Thus, a common regimen for a patient involves receiving a long acting basal insulin supplemented by a rapid acting insulin around mealtimes.

Insulin is a peptide hormone formed of two chains (A chain and B chain, respectively 21 and 30 amino acids in length) linked via disulfide bridges. Insulin normally exists at neutral pH in the form of a hexamer, each hexamer comprising three dimers bound together by zinc ions. Histidine residues on the insulin are known to be involved in the interaction with the zinc ions. Insulin is stored in the body in the hexameric form but the monomer form is the active form. Traditionally, therapeutic compositions of insulin have also been formulated in hexameric form in the presence of zinc ions. Typically, there are approximately three zinc cations per one insulin hexamer. It has been appreciated that the hexameric form is absorbed from the injection site considerably more slowly than the monomeric and dimeric form. Therefore, a faster onset of insulin action can be achieved if the hexameric form is destabilised allowing a more rapid dissociation of the zinc-bound hexamer into dimers and monomers in the subcutaneous space following injection. Three insulin analogues have been genetically engineered with this principle in mind. A first is insulin lispro (Humalog®) in which residues 28 and 29 of the B chain (Pro and Lys respectively) are reversed, a second is insulin aspart (NovoLog®) in which residue 28 of the B chain, normally Pro, is replaced by Asp and a third is insulin glulisine (Apidra®) in which residue 3 of the B chain, normally Asn, is replaced by Lys and residue 29 of the B chain, normally Lys, is replaced by Glu.

Whilst the existing rapid acting insulin analogues can achieve a more rapid onset of action, it has been appreciated that an even more rapid acting (“ultra rapid acting”) insulins can be achieved by removing the zinc cations from insulin altogether. Unfortunately, the consequence of the hexamer dissociation is typically a considerable impairment in insulin stability both with respect to physical stability (e.g. stability to aggregation) and chemical stability (e.g. stability to deamidation). For example, monomeric insulin or insulin analogues having a rapid onset of action are known to aggregate and become physically unstable very rapidly because the formulation of insoluble aggregates proceeds via monomers of insulin. Various approaches to addressing this problem have been described in the art:

U.S. Pat. No. 5,866,538 (Norup) describes insulin preparations of superior chemical stability comprising human insulin or an analogue or derivative thereof, glycerol and/or mannitol and 5 to 100 mM of a halogenide (e.g. NaCl).

U.S. Pat. No. 7,205,276 (Boderke) addresses the stability problems associated with preparing zinc free formulations of insulin and insulin derivatives and analogues and describes an aqueous liquid formulation comprising at least one insulin derivative, at least one surfactant, optionally at least one preservative and optionally at least one of an isotonicizing agent, a buffer and an excipient, wherein the formulation is stable and free from or contains less than 0.4% (e.g. less than 0.2%) by weight of zinc based on the insulin content of the formulation. The preferred surfactant appears to be polysorbate 20 (polyoxyethylene (20) sorbitan monolaurate).

US2008/0194461 (Maggio) describes formulations of peptides and polypeptides including insulin which contain an alkylglycoside, which component is said to reduce aggregation and immunogenicity.

WO2012/006283 (Pohl) describes formulations containing insulin together with a zinc chelator such as ethylenediaminetetraacetate (EDTA). Modulating the type and quantity of EDTA is said to change the insulin absorption profile. Calcium EDTA is the preferred form of EDTA since it is said to be associated with reduced pain at the injection site and is less likely to remove calcium from the body. Preferred formulations also contain citrate which is said to further enhance absorption and to improve the chemical stability of the formulation.

US2010/0227795 (Steiner) describes a composition comprising insulin, a dissociating agent such as citric acid or sodium citrate, and a zinc chelator such as EDTA wherein the formulation has a physiological pH and is a clear aqueous solution. The formulations are said to have improved stability and rapid onset of action.

WO2015/120457 (Wilson) describes stabilized ultra-rapid acting insulin formulations comprising insulin in combination with a zinc chelator such as EDTA, a dissolution/stabilization agent such as citric acid, a magnesium salt, a zinc compound and optionally additional excipients.

Further approaches to accelerating the absorption and effect of insulin through the use of specific accelerating additives have been described:

WO91/09617 (Jørgensen) reports that nicotinamide or nicotinic acid or a salt thereof increases the speed of absorption of insulin from aqueous preparations administered parenterally.

WO2010/149772 (Olsen) describes a formulation comprising insulin, a nicotinic compound and arginine. The presence of arginine is said to improve the chemical stability of the formulation.

WO2015/171484 (Christe) describes rapid acting formulations of insulin wherein onset of action and/or absorption of insulin is faster due to the presence of treprostinil.

US2013/0231281 (Soula) describes an aqueous solution composition comprising insulin or an insulin analogue and at least one oligosaccharide whose average degree of polymerisation is between 3 and 13 and whose polydispersity index is above 1.0, said oligosaccharide having partially substituted carboxyl functional groups, the unsubstituted carboxyl functional groups being salifiable. Such a formulation is said to be rapid acting.

It would be desirable if analogues or formulations of insulin were available which were ultra-rapid acting, thus more closely matching the activity of physiological insulin. There also remains a need in the art to provide further, and preferably improved, formulations of insulin and insulin analogues which are rapid acting and stable.

### SUMMARY OF THE INVENTION

According to the invention there is provided an aqueous liquid pharmaceutical formulation comprising (i) an insulin compound, (ii) ionic zinc, (iii) a nicotinic compound (iv) a non-ionic surfactant; and (v) a salt selected from the salts formed between Group 1 metals and a mono or divalent anion (“the formulation of the invention”).

The formulations of the invention provide insulin in a form which is rapid or ultra-rapid acting with good physical and chemical stability.

Formulations of the invention may be used in treatment of subjects suffering from diabetes mellitus, particularly Type 1 diabetes mellitus especially for administration at meal times.

### DESCRIPTION OF THE SEQUENCE LISTING

SEQ ID NO: 1: A chain of human insulin
SEQ ID NO: 2: B chain of human insulin
SEQ ID NO: 3: B chain of insulin lispro
SEQ ID NO: 4: B chain of insulin aspart
SEQ ID NO: 5: B chain of insulin glulisine

### DETAILED DESCRIPTION OF THE INVENTION

As used herein, “insulin compound” refers to insulin and insulin analogues.

As used herein, “insulin” refers to native human insulin having an A chain and a B chain as set out in SEQ ID NOs. 1 and 2 and containing and connected by disulfide bridges as in the native molecule (Cys A6-Cys A11, Cys B7 to Cys A7 and Cys-B19-Cys A20). Insulin is suitably recombinant insulin.

“Insulin analogue” refers to an analogue of insulin which is an insulin receptor agonist and has a modified amino acid sequence, such as containing 1 or 2 amino acid changes in the sequence of the A or B chain (especially the B chain). Desirably such amino acid modifications are intended to reduce affinity of the molecule for zinc and thus increase speed of action. Exemplary insulin analogues include faster acting analogues such as insulin lispro, insulin aspart and insulin glulisine. These forms of insulin have the human insulin A chain but variant B chains—see SEQ ID NOs. 3-5. Further faster acting analogues are described in EP0214826, EP0375437 and EP0678522 the contents of which are herein incorporated by reference in their entirety. Thus, desirably an insulin analogue has a speed of action which is the same as or preferably greater than that of insulin. The speed of action of insulin or an insulin analogue may be determined in the Diabetic Pig Pharmacokinetic/Pharmacodynamic Model (see Examples, General Methods).

In one embodiment the insulin compound is recombinant human insulin. In another embodiment it is insulin lispro. In another embodiment it is insulin aspart. In another embodiment it is insulin glulisine.

The term “nicotinic compound” refers to nicotinic acid and salts thereof and derivatives including esters and amides thereof such as nicotinamide. Exemplary salts of nicotinic acid include sodium, potassium, calcium and magnesium salts.

The term “aqueous pharmaceutical formulation”, as used herein, refers to a formulation suitable for therapeutic use in which the aqueous component is or comprises water, preferably distilled water, deionized water, water for injection, sterile water for injection or bacteriostatic water for injection. The aqueous pharmaceutical formulations of the invention are solution formulations in which all components are dissolved in water.

The term “monovalent or divalent anion” refers to an anion having one or more ionisable groups capable of being deprotonated in the formulation such that the anion has a charge of minus 1 or minus 2 and which anion does not contain any atoms or groups capable of being positively charged in the formulation. Thus, the scope of the term excludes all zwitterions and all amino acids. Other anions specifically excluded from the scope of this term include trivalent anions such as nitrate, citrate and phosphate.

The concentration of insulin compound in the formulation will typically be in the range 10-1000 U/ml, such as 50-500 U/ml e.g. 50-200 U/ml. An exemplary formulation contains insulin compound at a concentration of 100 U/ml (around 3.6 mg/ml). Another range of interest is 500-1000 U/ml e.g. 800-1000 U/ml and another exemplary formulation contains insulin compound at a concentration of 1000 U/ml (around 36 mg/ml).

The formulations of the invention contain ionic zinc i.e. Zn2+ ions. The source of the ionic zinc will typically be a water soluble zinc salt such as ZnCl2, ZnO, ZnSO4, Zn(NO3)2 or Zn(acetate)2 and most suitably ZnCl2 or ZnO.

The concentration of the ionic zinc in the formulation will typically be 0.05% or more e.g. 0.1% or more e.g. 0.2% or more, 0.3% or more or 0.4% or more by weight of zinc based on the weight of insulin compound in the formulation. Thus the concentration of the ionic zinc in the formulation may be 0.5% or more by weight of zinc based on the weight of insulin compound in the formulation, for example 0.5-1%, e.g. 0.5-0.75%, e.g. 0.5-0.6% by weight of zinc based on the weight of insulin compound in the formulation. For the purpose of the calculation the weight of the counter ion to zinc is excluded.

In a formulation e.g. containing 100 U/ml of insulin compound the concentration of the ionic zinc will typically be more than 0.015 mM e.g. more than 0.03 mM e.g. more than 0.06 mM, more than 0.09 mM or more than 0.12 mM. Thus concentration of the ionic zinc in the formulation may be more than 0.15 mM, for example 0.15-0.60 mM, e.g. 0.20-0.45 mM, e.g. 0.25-0.35 mM.

In a formulation e.g. containing 1000 U/ml of insulin compound the concentration of the ionic zinc will typically be more than 0.15 mM e.g. more than 0.3 mM e.g. more than 0.6 mM, more than 0.9 mM or more than 1.2 mM. Thus concentration of the ionic zinc in the formulation may be more than 1.5 mM, for example 1.5-6.0 mM, e.g. 2.0-4.5 mM, e.g. 2.5-3.5 mM.

The formulations of the invention comprise a nicotinic compound which is expected to increase the speed of onset of action of insulin formulated in formulations of the invention. In a preferred embodiment the nicotinic compound is nicotinamide. Alternatively it is nicotinic acid or a salt of nicotinic acid e.g. the sodium salt. Suitably, the concentration of nicotinic compound is in the range 10-150 mM, preferably in the range 20-100 mM, e.g. 50-100 mM such as around 80 mM.

The formulations of the invention contain a non-ionic surfactant.

A suitable class of non-ionic surfactants is the alkyl glycosides, especially dodecyl maltoside. Other alkyl glycosides include dodecyl glucoside, octyl glucoside, octyl maltoside, decyl glucoside, decyl maltoside, tridecyl glucoside, tridecyl maltoside, tetradecyl glucoside, tetradecyl maltoside, hexadecyl glucoside, hexadecyl maltoside, sucrose monooctanoate, sucrose mono decanoate, sucrose monododecanoate, sucrose monotridecanoate, sucrose monotetradecanoate and sucrose monohexadecanoate.

Another suitable class of non-ionic surfactants is the polysorbates (fatty acid esters of ethoxylated sorbitan), such as polysorbate 80 or polysorbate 20. Polysorbate 80 is polysorbate 80 is a mono ester formed from oleic acid and polyoxyethylene (20) sorbitan in which the number 20 indicates the number of oxyethylene groups in the molecule. Polysorbate 80 is known under a range of brand names including in particular Tween 80, and also Alkest TW 80. Polysorbate 20 is a mono ester formed from lauric acid and polyoxyethylene (20) sorbitan in which the number 20 indicates the number of oxyethylene groups in the molecule. Polysorbate 20 is known under a range of brand names including in particular Tween 20, and also Alkest TW 20. Other suitable polysorbates include polysorbate 40 and polysorbate 60.

Another suitable class of non-ionic surfactants is block copolymers of polyethylene glycol and polypropylene glycol, also known as poloxamers, especially poloxamer 188, poloxamer 407, poloxamer 171 and poloxamer 185. Poloxamers are also known under brand names Pluronics or Koliphors. For example, poloxamer 188 is marketed as Pluronic F-68.

Another suitable class of non-ionic surfactants is alkyl ethers of polyethylene glycol, especially those known under a brand name Brij, such as selected from polyethylene glycol (2) hexadecyl ether (Brij 52), polyethylene glycol (2) oleyl ether (Brij 93) and polyethylene glycol (2) dodecyl ether (Brij L4). Other suitable Brij surfactants include polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Another suitable class of non-ionic surfactants are alkylphenyl ethers of polyethylene glycol, especially 4-(1,1,3,3-tetramethylbutyl)phenyl-polyethylene glycol, also known under a brand name Triton X-100.

Particularly suitable are non-ionic surfactants with molecular weight of less than 1000 g/mole, especially less than 600 g/mole, such as 4-(1,1,3,3-tetramethylbutyl)phenyl-polyethylene glycol (Triton X-100) (647 g/mole), dodecyl maltoside (511 g/mole), octyl glucoside (292 g/mole), polyethylene glycol (2) dodecyl ether (Brij L4) (362 g/mole), polyethylene glycol (2) oleyl ether (Brij 93) (357 g/mole) and polyethylene glycol (2) hexadecyl ether (Brij 52) (330 g/mole).

The concentration of the non-ionic surfactant in the formulation will typically be in the range 1-1000 μg/ml, e.g. 5-500 μg/ml, e.g. 10-200 μg/ml, such as 10-100 μg/ml especially around 50 μg/ml.

The formulations of the invention comprise a salt selected from the salts formed between Group 1 metals and mono or divalent anions. Suitable Group 1 metals include sodium and potassium, especially sodium. Anions are preferably monovalent anions. Anions may be inorganic or organic however are preferably inorganic. Example inorganic anions include halides such as chloride or bromide (preferably chloride) and sulfate. Example organic anions include ions derived from mono or divalent carboxylic acids especially monocarboxylic acids such as acetate and benzoate and dicarboxylic acids such as succinate, maleate and malate. A preferred organic anion is acetate. Exemplary salts include sodium chloride, potassium chloride and sodium acetate. The preferred salt is sodium chloride.

The salt selected from the salts formed between Group 1 metals and mono or divalent anions may suitably be present in the formulation at a concentration of 30-200 mM e.g. 50-200 mM e.g. 50-120 mM e.g. 65-75 mM e.g. around 70 mM.

Suitably the pH of the aqueous formulations of the invention is in the range 5.5-9.0 especially 6.5-8.0 e.g. 7.0-7.5. In order to minimise injection pain the pH is preferably close to physiological pH (around pH 7.4). Another pH range of interest is 7.6-8.0 e.g. around 7.8.

Optionally, the formulation of the invention comprises a buffer in order to stabilise the pH of the formulation, which can also be selected to enhance protein stability. In one embodiment, a buffer is selected to have a pKa close to the pH of the formulation; for example histidine is suitably employed as a buffer when the pH of the formulation is in the range 5.0-7.0. Such a buffer may be employed in a concentration of 0.5-20 mM e.g. 2-5 mM. As another example, phosphate is suitably employed as a buffer when the pH of the formulation is in the range 6.1-8.1. Such a buffer may be employed in a concentration of 0.5-20 mM e.g. 2-5 mM. Another possible buffer is citrate. Alternatively, in another embodiment, the formulation of the invention is further stabilised as disclosed in WO2008/084237 (herein incorporated in its entirety by reference), which describes a formulation comprising a protein and one or more additives, characterised in that the system is substantially free of a conventional buffer, i.e. a compound with an ionisable group having a pKa within 1 unit of the pH of the formulation at the intended temperature range of storage of the formulation, such as 25° C. In this embodiment, the pH of the formulation is set to a value at which the formulation has maximum measurable stability with respect to pH; the one or more additives (displaced buffers) are capable of exchanging protons with the insulin compound and have pKa values at least 1 unit more or less than the pH of the formulation at the intended temperature range of storage of the formulation. The additives may have ionisable groups having pKa between 1 to 5 pH units, preferably between 1 to 3 pH units, most preferably from 1.5 to 2.5 pH units, of the pH of the aqueous formulation at the intended temperature range of storage of the formulation (e.g. 25° C.). Such additives may typically be employed at a concentration of 0.5-10 mM e.g. 2-5 mM.

The aqueous formulations of the present invention cover a wide range of osmolarity, including hypotonic, isotonic and hypertonic formulations. Preferably, the formulations of the invention are substantially isotonic. Suitably the osmolarity of the formulation is selected to minimize pain according to the route of administration e.g. upon injection. Preferred formulations have an osmolarity in the range of about 200 to about 500 mOsm/L. Preferably, the osmolarity is in the range of about 250 to about 350 mOsm/L. More preferably, the osmolarity is about 300 mOsm/L.

The presence of the salt will modify the tonicity of the formulation, nevertheless, tonicity of the formulation may be further adjusted with an uncharged tonicity modifying agent. Examples of uncharged tonicity modifying agents include sugars, sugar alcohols and other polyols, such as trehalose, sucrose, mannitol, glycerol, 1,2-propanediol, raffinose, lactose, dextrose, sorbitol or lactitol (especially trehalose, mannitol, glycerol or 1,2-propanediol, particularly glycerol). Uncharged tonicity modifying agent is preferably used at a concentration of 20-200 mM, e.g. 50-150 mM, e.g. around 80 mM.

The ionic strength of a formulation may be calculated according to the formula:

$$I = 0.5 \times {\sum }_{X = 1}^{n} {c}_{x} {z}_{x}^{2}$$

in which cₓ is molar concentration of ion x (mol L⁻¹), zₓ is the absolute value of the charge of ion x and the sum covers all ions (n) present in the formulation. The contribution of the insulin compound itself should be ignored for the purposes of the calculation. For zwitterions the absolute value of the charge is the total charge excluding polarity, e.g. for glycine the possible ions have absolute charge of 0, 1 or 2 and for aspartate the possible ions have absolute charge of 0, 1, 2 or 3.

In general, the ionic strength of the formulation is suitably in the range of around 30 mM up to around 500 mM.

When the insulin compound is insulin lispro, the ionic strength of the formulation is suitably kept to a minimum level since higher ionic strength formulations are less stable than lower ionic strength formulations. Suitably the ionic strength taking account of ions in the formulation except for the zinc binding species and the insulin compound is less than 60 mM, e.g. less than 50 mM, e.g. less than 40 mM such as 30-40 mM.

When the insulin compound is insulin aspart at a concentration of >500 U/ml (e.g. 1000 U/ml), the ionic strength of the formulation is suitably kept to a minimum level since higher ionic strength formulations are less stable than lower ionic strength formulations. Suitably the ionic strength taking account of ions in the formulation except for the zinc binding species and the insulin compound is less than 60 mM, e.g. less than 50 mM, e.g. less than 40 mM such as 30-40 mM.

When the insulin compound is insulin aspart at a concentration of 500 U/ml or less (e.g. 100 U/ml), the ionic strength of the formulation may be high. Suitably the ionic strength taking account of ions in the formulation except for the zinc binding species and the insulin compound is more than 50 mM, e.g. more than 100 mM, e.g. 50-500 mM or 100-500 mM or 100-300 mM such as around 150 mM.

The formulations of the invention can optionally include preservative, preferably phenol, m-cresol, chlorocresol, benzyl alcohol, propylparaben, methylparaben, benzalkonium chloride or benzethonium chloride.

Formulations of the invention may optionally include other beneficial components including stabilising agents.

In a first embodiment, the formulations of the invention comprise zinc binding species. Zinc binding species should be capable of complexing ionic zinc and will be selected from species having a log K metal binding stability constant with respect to zinc ion binding of 4.5 or more (e.g. 4.5-12.3 or 4.5-10) as determined at 25° C. Metal binding stability constants listed in the National Institute of Standards and Technology reference database 46 (Critically Selected Stability Constants of Metal Complexes) can be used. The database typically lists log K constants determined at 25° C. Therefore, the suitability of a zinc binding species to be optionally included in formulations of the invention can be determined based on its log K metal binding stability constant with respect to zinc binding, as measured at 25° C. and as quoted by the database. Exemplary zinc binding species having a log K with respect to zinc ion binding of 4.5 or more to be optionally included include polydendate organic anions. Exemplary zinc binding species having a log K with respect to zinc ion binding of 4.5 or more to be optionally included include those having a log K with respect to zinc ion binding of 4.5-10 include citrate (log K=4.93) which can, for example, be employed as sodium citrate. Further examples include pyrophosphate (log K=8.71), aspartate (log K=5.87), glutamate (log K=4.62), cysteine (log K=9.11), cystine (log K=6.67) and glutathione (log K=7.98). Other possible zinc binding species include substances that can contribute a lone pair of electrons or electron density for interaction with ionic zinc such as polydendate amines including ethylenediamine (log K=5.69), diethylenetriamine (DETA, log K=8.88) and aromatic or heteroaromatic substances that can contribute a lone pair of electrons especially those comprising an imidazole moiety such as histidine (log K=6.51). Exemplary zinc binding species having a log K with respect to zinc ion binding of 4.5 or more include those having a log K with respect to zinc ion binding of more than 10 such as triethylenetetramine (TETA, log K=11.95) and ethylenediaminetetracetate (EDTA, log K=14.5). Suitably, the zinc binding species have a log K with respect to zinc ion binding of 4.5-12.3 e.g. 4.5-10, such as citrate.

Reference to citrate, pyrophosphate, glutamate, ethylenediaminetetracetate etc. refers to the corresponding acid or an ionised form of the corresponding acid such as citric acid, pyrophosphoric acid, glutamic acid, ethylenediaminetetracetic acid etc.

Zinc ion binding species which have acid forms (e.g. citric acid) may be introduced into the aqueous formulations of the invention in the form of a salt of the acid, such as a sodium salt (e.g. sodium citrate). Alternatively, they can be introduced in the form of the acid with subsequent adjustment of pH to the required level.

Formulations which comprise zinc binding species selected from species having a log K with respect to zinc ion binding of 4.5 or more at 25° C. e.g. may do so at a concentration of at least 1 mM, such as at least 2 mM or at least 5 mM. For example, the concentration of the zinc binding species in the formulation in the formulation may typically be in the range 1-50 mM, more preferably 5-50 mM e.g. 10-50 mM e.g. 10-30 mM, more preferably around 20 mM (e.g. 22 mM), especially when the zinc binding species is citrate or histidine and especially for insulin compound 100 U/ml formulations. Suitably the concentration of the zinc binding species in the formulation is 10-50 mM e.g. 30-50 mM e.g. 40-50 mM, more preferably around 44 mM when the zinc binding species is citrate or histidine for insulin compound 1000 U/ml formulations. In an embodiment, the concentration of the zinc binding species is 10 mM or more. Anionic zinc binding species may be employed as the free acid or a salt form, such as a salt form with sodium or calcium ions, especially sodium ions. A mixture of zinc binding species may be employed, although a single zinc binding species is preferred.

The molar ratio of ionic zinc to zinc binding species in the formulation may be in the range 1:1 to 1000 e.g.1:1 to 1:500 e.g. 1:1 to 1:250 or 1:3 to 1:500 e.g.1:3 to 1.175.

For example, a suitable molar ratio of ionic zinc to zinc binding species is 1:10-1:500 e.g. 1:20-1:500 e.g. 1:20-1:100 or 1:40-1:250, e.g. 1:40-1:90 or 1:60-1:200, e.g. 1:60-1:80, especially for citrate or histidine as zinc binding species. The following ranges are particularly of interest especially for citrate or histidine as zinc binding species: 1:10-1:500 e.g. 1:10-1:200 e.g. 1:10 to 1:100 e.g. 1:10-1:50, e.g. 1:10 to 1:30 (especially for insulin compound 1000 U/ml formulation) or 1:50-1:100, e.g. 1:60-1:80 (especially for insulin compound 100 U/ml formulation).

For example, a formulation containing 100 U/ml of insulin compound may contain around 0.3 mM of ionic zinc (i.e. around 19.7 μg/ml of ionic zinc, i.e. around 0.54% by weight of zinc based on the weight of insulin compound in the formulation) and around 15-30 mM e.g. 20-30 mM zinc binding species (especially citrate).

For example, a formulation containing 1000 U/ml of insulin compound may contain around 3 mM of ionic zinc (i.e. around 197 μg/ml of ionic zinc, i.e. around 0.54% by weight of zinc based on the weight of insulin compound in the formulation) and around 30-60 mM e.g. 40-60 mM zinc binding species (especially citrate).

In an alternative embodiment, the formulations of the invention are free of zinc binding species selected from species having a log K with respect to zinc ion binding of 4.5 or more at 25° C. or contain a concentration of zinc binding species selected from species having a log K with respect to zinc ion binding of 4.5 or more at 25° C. which is less than 1 mM e.g. less than 0.5 mM. For example, the formulations are substantially free of or free of zinc binding species selected from species having a log K with respect to zinc ion binding of 4.5 or more at 25° C. “Substantially free” in this context means that the concentration of zinc binding species selected from species having a log K with respect to zinc ion binding of 4.5 or more at 25° C. is less than 0.1 mM, such as less than 0.05 mM or less than 0.04 mM or less than 0.01 mM.

The formulations of the invention may be substantially free of zinc binding species selected from species having a log K with respect to zinc ion binding of more than 10 e.g. more than 12.3 at 25° C. for example are substantially free of EDTA. “Substantially free” in this context means that the concentration of zinc binding species referred to is less than 0.1 mM, such as less than 0.05 mM or less than 0.04 mM or less than 0.01 mM.

In an embodiment of the invention the formulations are free of amino acids such as glutamic acid and are also free of the corresponding ionic forms of these acids.

In an embodiment of the invention the formulations are free of arginine.

In an embodiment of the invention the formulations are free of protamine and protamine salts.

In an embodiment of the invention the formulations are free of magnesium ions.

In an embodiment of the invention the formulations are free of calcium ions.

In an embodiment of the invention the formulations are free of mannitol.

In an embodiment of the invention the formulations are free of glycerol.

Suitably the formulations of the invention are sufficiently stable that the concentration of high molecular weight species remains low upon extended storage. The term “high molecular weight species” as used herein, refers to any irreversibly formed component of the protein content which has an apparent molecular weight at least about double the molecular weight of the parent insulin compound, as detected by a suitable analytical method, such as size-exclusion chromatography. That is, high molecular weight species are multimeric aggregates of the parent insulin compound. The multimeric aggregates may comprise the parent protein molecules with considerably altered conformation or they may be an assembly of the parent protein units in the native or near-native conformation. The determination of high molecular weight species can be done using methods known in the art, including size exclusion chromatography, electrophoresis, analytical ultracentrifugation, light scattering, dynamic light scattering, static light scattering and field flow fractionation.

Suitably the formulations of the invention are sufficiently stable that they remain substantially free of visible particles after storage at 30° C. for at least one, two or three months. Visible particles are suitably detected using the 2.9.20. European Pharmacepoeia Monograph (Particulate Contamination: Visible Particles).

Suitably the formulations of the invention are sufficiently stable that the concentration of related species remains low upon extended storage. The term “related species” as used herein, refers to any component of the protein content formed by a chemical modification of the parent insulin compound, particularly desamido or cyclic imide forms of insulin. Related species are suitably detected by RP-HPLC.

In a preferred embodiment, the formulation of the invention retains at least 95%, e.g. at least 96%, e.g. at least 97%, e.g. at least 98%, e.g. at least 99% parent insulin compound (by weight of total protein) after storage at 30° C. for one, two or three months. The percentage of insulin compound (by weight of total protein) may be determined by size-exclusion chromatography or RP-HPLC.

In a preferred embodiment, the formulation of the invention comprises no more than 4% (by weight of total protein), preferably no more than 2% high molecular weight species after storage at 30° C. for one, two or three months.

In a preferred embodiment, the formulation of the invention comprises no more than 4% (by weight of total protein), preferably no more than 2%, preferably no more than 1% A-21 desamido form of the insulin compound after storage at 30° C. for one, two or three months.

In preferred embodiments, a formulation of the present invention should exhibit an increase in high molecular weight species during storage which is at least 10% lower, preferably at least 25% lower, more preferably at least 50% lower, than a formulation lacking the non-ionic surfactant but otherwise identical, following storage under the same conditions (e.g. 30° C.) and length of time (e.g. one, two or three months).

In preferred embodiments, a formulation of the present invention should exhibit an increase in related species during storage which is at least 10% lower, preferably at least 25% lower, more preferably at least 50% lower, than a formulation lacking the non-ionic surfactant but otherwise identical, following storage under the same conditions (e.g. 30° C.) and length of time (e.g. one, two or three months).

The speed of action of a formulation of the invention may be determined in the Diabetic Pig Pharmacokinetic/Pharmacodynamic Model (see Examples, General Methods). In preferred embodiments, a formulation of the present invention should exhibit a Tmax (i.e. time to peak insulin concentration) that is at least 10% shorter, preferably at least 20% shorter, more preferably at least 30% shorter than a formulation lacking the nicotinic compound but otherwise identical, using the model. In preferred embodiments, a formulation of the present invention should exhibit an area under the curve on the pharmacodynamics profile within the first 45 minutes after injection that is at least 10% greater, preferably at least 20% greater, more preferably at least 30% greater than a formulation lacking the nicotinic compound but otherwise identical, using the model.

According to further aspects of the invention, there is provided a formulation of the invention for use in the treatment of a subject suffering from diabetes mellitus. There is also provided a method of treatment of diabetes mellitus which comprises administering to a subject in need thereof an effective amount of a formulation of the invention.

A typical dose of the formulation of the invention is 2-30 U, e.g. 5-15 U. Administration should suitably occur in the window between 15 minutes before eating (i.e. before start of a meal) and 15 minutes after eating (i.e. after end of a meal).

An aspect of the invention is a container e.g. made of plastics or glass containing one dose or a plurality of doses of the formulation of the invention. The container can, for example, be a cartridge designed to be a replaceable item for use with an injection device.

The formulations of the invention may suitably be packaged for injection, especially sub-cutaneous or intramuscular injection. Sub-cutaneous injection is preferred. Injection may be by conventional syringe or more preferably via a pen device adapted for use by diabetic subjects. Exemplary pen devices include the Kwikpen® device and the Flexpen® device.

An aspect of the invention is an injection device, particularly a device adapted for subcutaneous or intramuscular injection, for single or multiple use comprising a container containing one dose or a plurality of doses of the formulation of the invention together with an injection needle. In an embodiment the container is a replaceable cartridge which contains a plurality of doses. In an embodiment, the needle is replaceable e.g. after each occasion of use.

Another aspect of the invention is a medical device comprising a reservoir comprising plurality of doses of the formulation of the invention and a pump adapted for automatic or remote operation such that upon automatic or remote operation one or more doses of the formulation of the invention is administered to the body e.g. subcutaneously or intramuscularly. Such devices may be worn on the outside of the body or implanted in the body.

Formulations of the invention may be prepared by mixing the ingredients. For example, the insulin compound may be dissolved in an aqueous formulation comprising the other components. Alternatively, the insulin compound may be dissolved in a strong acid (typically HCl), after dissolution diluted with an aqueous formulation comprising the other components, and then pH adjusted to the desired pH with addition of alkali (e.g. NaOH). As a variation on this method, a step of neutralising the acid solution may be performed before the dilution step and it may then not be necessary to adjust the pH after the dilution step (or a small adjustment only may be necessary).

According to another aspect of the invention there is provided a dry solid pharmaceutical composition suitable for reconstitution with an aqueous medium which comprises (i) an insulin compound, (ii) ionic zinc, (iii) a nicotinic compound, (iv) a non-ionic surfactant; and (v) a salt selected from the salts formed between Group 1 metals and a mono or divalent anion. Thus a formulation of the invention may be prepared by dissolving such a dry solid pharmaceutical composition in an aqueous medium e.g. water or saline. Such a dry solid pharmaceutical composition may be prepared by dehydrating (e.g. freeze drying) a formulation of the invention. The invention also provides a container containing one dose or a plurality of doses of such a dry solid pharmaceutical composition.

Further aspects of the invention include:

A method of improving the storage stability of an aqueous liquid pharmaceutical formulation comprising (i) an insulin compound, (ii) ionic zinc, (iii) a nicotinic compound and (iv) a salt selected from the salts formed between Group 1 metals and a mono or divalent anion which comprises adding a non-ionic surfactant to the formulation;

Use of a non-ionic surfactant to improve the storage stability of an aqueous liquid pharmaceutical formulation comprising (i) an insulin compound, (ii) ionic zinc, (iii) a nicotinic compound and (iv) a salt selected from the salts formed between Group 1 metals and a mono or divalent anion;

A method of improving the storage stability of an aqueous liquid pharmaceutical formulation comprising (i) an insulin compound, (ii) ionic zinc and (iii) a nicotinic compound which comprises adding a non-ionic surfactant and a salt selected from the salts formed between Group 1 metals and a mono or divalent anion to the formulation; and

Use of a non-ionic surfactant and a salt selected from the salts formed between Group 1 metals and a mono or divalent anion to improve the storage stability of an aqueous liquid pharmaceutical formulation comprising (i) an insulin compound, (ii) ionic zinc and (iii) a nicotinic compound.

Formulations of the invention are expected to have one or more of the following advantageous properties:
rapid speed of action, typically faster than normal human insulin, upon administration to a subject;
good physical stability upon storage, especially as measured by the amount of HMWS or visual detection of particles;
good chemical stability upon storage, especially as measured by the amount of related products e.g. products of deamidation.

Further aspects of the invention are illustrated by the following clauses:
Clause 1. An aqueous liquid pharmaceutical formulation comprising (i) an insulin compound, (ii) ionic zinc, (iii) a nicotinic compound (iv) a non-ionic surfactant; and (v) a salt selected from the salts formed between Group 1 metals and a mono or divalent anion.
Clause 2. The formulation according to clause 1 wherein the insulin compound is insulin lispro.
Clause 3. The formulation according to clause 1 wherein the insulin compound is insulin aspart.
Clause 4. The formulation according to clause 1 wherein the insulin compound is insulin glulisine.
Clause 5. The formulation according to clause 1 wherein the insulin compound is recombinant human insulin.
Clause 6. The formulation according to any one of clauses 1 to 5, wherein the insulin compound is present at a concentration of 10-1000 U/ml.
Clause 7. The formulation according to any one of clauses 1 to 6, wherein the nicotinic compound is nicotinamide.
Clause 8. The formulation according to any one of clauses 1 to 6, wherein the nicotinic compound is nicotinic acid or a salt thereof.
Clause 9. The formulation according to any one of clauses 1 to 8, wherein the nicotinic compound is present at a concentration of 10-150 mM.
Clause 10. The formulation according to any one of clauses 1 to 9 wherein the non-ionic surfactant is an alkyl glycoside.
Clause 11. The formulation according to clause 10 wherein the alkyl glycoside is dodecyl maltoside.
Clause 12. The formulation according to any one of clauses 1 to 9 wherein the non-ionic surfactant is a polysorbate surfactant.
Clause 13. The formulation according to clause 12 wherein the polysorbate surfactant is polysorbate 20 or polysorbate 80.
Clause 14. The formulation according to any one of clauses 1 to 9 wherein the non-ionic surfactant is an alkyl ether of polyethylene glycol.
Clause 15. The formulation according to clause 14 wherein the alkyl ether of polyethylene glycol is selected from polyethylene glycol (2) dodecyl ether, polyethylene glycol (2) oleyl ether and polyethylene glycol (2) hexadecyl ether.
Clause 16. The formulation according to any one of clauses 1 to 9 wherein the non-ionic surfactant is a block copolymer of polyethylene glycol and polypropylene glycol.
Clause 17. The formulation according to clause 16 wherein the block copolymer of polyethylene glycol and polypropylene glycol is poloxamer 188, poloxamer 407, poloxamer 171 or poloxamer 185.
Clause 18. The formulation according to any one of clauses 1 to 9 wherein the non-ionic surfactant is an alkylphenyl ether of polyethylene glycol.
Clause 19. The formulation according to clause 18 wherein the alkylphenyl ether of polyethylene glycol is 4-(1,1,3,3-tetramethylbutyl)phenyl-polyethylene glycol.
Clause 20. The formulation according to any one of clauses 1 to 19 wherein the surfactant is present at a concentration of 1-1000 μg/ml.
Clause 21. The formulation according to any clause 20 wherein the surfactant is present at a concentration of 10-100 μg/ml.
Clause 22. The formulation according to any one of clauses 1 to 21 wherein the salt selected from the salts formed between Group 1 metals and a mono or divalent anion is a sodium salt of a mono or divalent anion.
Clause 23. The formulation according to any one of clauses 1 to 22 wherein the anion is a monovalent anion.
Clause 24. The formulation according to any one of clauses 1 to 23 wherein the anion is an inorganic anion.
Clause 25. The formulation according to any one of clauses 1 to 23 wherein the anion is an organic anion.
Clause 26. The formulation according to clause 24 wherein the anion is chloride.
Clause 27. The formulation according to clause 25 wherein the anion is acetate.
Clause 28. The formulation according to any one of clauses 1 to 27 wherein the salt selected from the salts formed between Group 1 metals and mono or divalent anions is present in the formulation at a concentration of 30-200 mM
Clause 29. The formulation according to any one of clauses 1 to 28, wherein ionic zinc is present in the formulation at a concentration of 0.05% or more by weight of zinc based on the weight of insulin compound in the formulation.
Clause 30. The formulation according to clause 29, wherein the ionic zinc is present at a concentration of 0.5-1% by weight of zinc based on the weight of insulin compound in the formulation.
Clause 31. The formulation according to any one of clauses 1 to 30 comprising an uncharged tonicity modifying agent.
Clause 32. The formulation according to clause 31, wherein the uncharged tonicity modifying agent is selected from the group consisting of trehalose, mannitol, glycerol or 1,2-propanediol.
Clause 33. The formulation according to clause 32, wherein the uncharged tonicity modifying agent is glycerol.
Clause 34. The formulation according to any one of clauses 1 to 33, wherein the formulation is isotonic.
Clause 35. The formulation according to any one of clauses 1 to 34, wherein the pH is in the range 5.5 to 9.0.
Clause 36. The formulation according to any of clauses 1 to 35, comprising a preservative.
Clause 37. The formulation according to clause 36, wherein the preservative is selected from the group consisting of phenol, m-cresol, chlorocresol, benzyl alcohol, propylparaben, methylparaben, benzalkonium chloride and benzethonium chloride.
Clause 38. The formulation according to any one of clauses 1 to 37 comprising zinc binding species selected from species having a log K with respect to zinc ion binding of 4.5 or more at 25° C.
Clause 39. The formulation according to any one of clauses 1 to 37 which is substantially free of zinc binding species selected from species having a log K with respect to zinc ion binding of 4.5 or more at 25° C.
Clause 40. A formulation according to any one of clauses 1 to 39 for use in the treatment of a subject suffering from diabetes mellitus.
Clause 41. A method of treatment of diabetes mellitus which comprises administering to a subject in need thereof an effective amount of a formulation according to any one of clauses 1 to 39.
Clause 42. A container containing one dose or a plurality of doses of the formulation according to any one of clauses 1 to 39.
Clause 43. An injection device for single or multiple use comprising a container containing one dose or a plurality of doses of the formulation according to any one of clauses 1 to 39 together with an injection needle.
Clause 44. A medical device comprising a reservoir comprising plurality of doses of the formulation according to any one of clauses 1 to 39 and a pump adapted for automatic or remote operation such that upon automatic or remote operation one or more doses of the formulation is administered to the body.
Clause 45. A dry solid pharmaceutical composition suitable for reconstitution with an aqueous medium which comprises (i) an insulin compound, (ii) ionic zinc, (iii) a nicotinic compound (iv) a non-ionic surfactant; and (v) a salt selected from the salts formed between Group 1 metals and a mono or divalent anion.
Clause 46. A method of preparing a formulation according to any one of clauses 1 to 39 which comprises dissolving a dry solid pharmaceutical composition according to clause 44 in an aqueous medium.
Clause 47. A method of improving the storage stability of an aqueous liquid pharmaceutical formulation comprising (i) an insulin compound, (ii) ionic zinc, (iii) a nicotinic compound and (iv) a salt selected from the salts formed between Group 1 metals and a mono or divalent anion which comprises adding a non-ionic surfactant to the formulation.
Clause 48. Use of a non-ionic surfactant to improve the storage stability of an aqueous liquid pharmaceutical formulation comprising (i) an insulin compound, (ii) ionic zinc, (iii) a nicotinic compound and (iv) a salt selected from the salts formed between Group 1 metals and a mono or divalent anion.
Clause 49. A method of improving the storage stability of an aqueous liquid pharmaceutical formulation comprising (i) an insulin compound, (ii) ionic zinc and (iii) a nicotinic compound which comprises adding a non-ionic surfactant and a salt selected from the salts formed between Group 1 metals and a mono or divalent anion to the formulation.
Clause 50. Use of a non-ionic surfactant and a salt selected from the salts formed between Group 1 metals and a mono or divalent anion to improve the storage stability of an aqueous liquid pharmaceutical formulation comprising (i) an insulin compound, (ii) ionic zinc and (iii) a nicotinic compound.

Abbreviations
EDTA ethylenediaminetetraacetate
EGTA ethyleneglycoltetraacetate
DETA diethylenetriamine
TETA triethylenetetramine
HPLC high performance liquid chromatography
BMWS high molecular weight species
RP reverse phase
SEC size-exclusion chromatography
PD pharmacodynamic

### EXAMPLES

### General Methods

### (a) The Diabetic Pig Pharmacokinetic/Pharmacodynamic Model: Method for Determining Speed of Action:

10 male diabetic Yucatan miniature pigs are used. Pigs are injected subcutaneously with a sample of the test formulation and blood is taken (1 or 2 ml) at the following time-points (min) with respect to the injection: −30 (or −15), 0, 5, 10, 15, 20, 30, 40, 50, 60, 75, 90, 105, 120, 150, 180, 210 and 240. For pharmacodynamics profile, serum is analysed for glucose (using a commercially available glucometer). For pharmacokinetic profile, insulin concentration is determined in the serum using an immunoassay.

### (b) Visual Assessment

Visible particles are suitably detected using the 2.9.20. European Pharmacepoeia

Monograph (Particulate Contamination: Visible Particles). The apparatus required consists of a viewing station comprising:
a matt black panel of appropriate size held in a vertical position
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded.

The visual scores are ranked as follows:
Visual score 1: Clear solution, virtually free of particles
Visual score 2: ˜ 5 very small particles
Visual score 3: ˜ 10-20 very small particles
Visual score 4: 20-50 particles, including larger particles
Visual score 5: >50 particles, including larger particles

Whilst the particles in samples with visual scores 4 and 5 are clearly detectable on casual visual assessment under normal light, samples with visual score 1-3 generally appear as clear solutions on the same assessment. Samples with visual scores 1-3 are considered to be “Pass”; samples with visual score 4-5 are considered to be “Fail”

### (c) Size Exclusion Chromatography

Ultra-high performance size exclusion chromatography of insulin preparations was performed using the Waters ACQUITY H-class Bio UPLC® system with a 1.7 μm Ethylene Bridged Hybrid 125 Å pore packing material in a 300 mm by 4.6 mm column. The column was equilibrated in 0.65 mg/ml L-arginine, 20% v/v acetonitrile, 15% v/v glacial acetic acid mobile phase and 10 μl of sample, acidified with 0.01M HCl, was analysed at 0.4 mL/min, with 276 nm UV detection. All analyses were performed at ambient temperature. Results are expressed as % high molecular weight species (HMWS) with respect to the total protein content.

### (d) Reversed-Phase Chromatography

Ultra-high performance reverse phase chromatography was performed using the Waters ACQUITY H-class Bio UPLC® system with a 1.7 μm Ethyene Bridged Hybrid particle, 130 Å pore resin trifunctionally immobilised with a C18 ligand in a 50 mm by 2.1 mm column. Insulin samples were bound in a 82% w/v Na₂SO₄, 18% v/v acetonitrile, pH 2.3 mobile phase and eluted in 50% w/v Na₂SO₄, 50% v/v acetonitrile gradient flow. 2μl of sample was acidified with 0.01M HCl and analysed at 0.61 mL/min, with 214 nm UV detection. All analyses were performed at 40° C.

### Example 1

### Example Formulations

The following example formulations may be prepared:

### Example A

| Insulin compound* | 100 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl₂) | the weight of insulin compound in the formulation | |
| Nicotinamide | 80 | mM |
| NaCl | 70 | mM |
| Dodecyl maltoside | 0.1 | mM |

| | |
|---|---|
| Water for injection | qs |
| Residual NaCl | Acidification and subsequent neutralisation during |
| | preparation results in formation of 2-4 mM NaCl |
| pH | Adjusted to 7.4 |

### Example B

| Insulin compound* | 100 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Dodecyl maltoside | 0.1 | mM |

| | |
|---|---|
| Water for injection | qs |
| Residual NaCl | Acidification and subsequent neutralisation during |
| | preparation results in formation of 2-4 mM NaCl |
| pH | Adjusted to 7.8 |

### Example C

| Insulin compound* | 1000 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Dodecyl maltoside | 0.05 | mM |

| | |
|---|---|
| Water for injection | qs |
| Residual NaCl | Acidification and subsequent neutralisation during |
| | preparation results in formation of 2-4 mM NaCl |
| pH | Adjusted to 7.4 |

### Example D

| Insulin compound* | 1000 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Dodecyl maltoside | 0.05 | mM |

| | |
|---|---|
| Water for injection | qs |
| Residual NaCl | Acidification and subsequent neutralisation during |
| | preparation results in formation of 2-4 mM NaCl |
| pH | Adjusted to 7.8 |

### Example E

| Insulin compound* | 100 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Polysorbate 80 | 0.05 | mg/ml |

| | |
|---|---|
| Water for injection | qs |
| Residual NaCl | Acidification and subsequent neutralisation during |
| | preparation results in formation of 2-4 mM NaCl |
| pH | Adjusted to 7.4 |

### Example F

| Insulin compound* | 1000 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Polysorbate 80 | 0.05 | mg/ml |

| | |
|---|---|
| Water for injection | qs |
| Residual NaCl | Acidification and subsequent neutralisation during |
| | preparation results in formation of 2-4 mM NaCl |
| pH | Adjusted to 7.4 |

### Example G

| Insulin compound* | 100 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Polysorbate 20 | 0.05 | mg/ml |

| | |
|---|---|
| Water for injection | qs |
| Residual NaCl | Acidification and subsequent neutralisation during |
| | preparation results in formation of 2-4 mM NaCl |
| pH | Adjusted to 7.4 |

### Example H

| Insulin compound* | 1000 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| NaCl | 70 | mM |
| Polysorbate 20 | 0.05 | mg/ml |

| | |
|---|---|
| Water for injection | qs |
| Residual NaCl | Acidification and subsequent neutralisation during |
| | preparation results in formation of 2-4 mM NaCl |
| pH | Adjusted to 7.4 |

### Example I

| Insulin compound* | 100 | U/ml |
|---|---|---|
| Sodium phosphate | 2 | mM |
| phenol | 15.9 | mM |
| m-cresol | 15.9 | mM |
| Ionic zinc (as | 19.7 μg/ml (0.3 mM), equals 0.55% (w/w) based on | |
| ZnCl2) | the weight of insulin compound in the formulation | |
| Nicotinamide | 80 | mM |
| Citric acid | 22 | mM |
| Glycerol | 70 | mM |
| Dodecyl maltoside | 0.1 | mM |

| | |
|---|---|
| Water for injection | qs |
| Residual NaCl | Acidification and subsequent neutralisation during |
| | preparation results in formation of 2-4 mM NaCl |
| pH | Adjusted to 7.4 |

Examples A to I: * Insulin compound=insulin aspart or insulin lispro or insulin glulisine or recombinant human insulin

### Method for Preparation for the Above Formulations:

Insulin powder is added to water and HCl is added until the powder is fully dissolved (pH has to be <3 in order to achieve full dissolution). ZnCl₂ is added to the required level. Once dissolved, pH is adjusted to approximately 7 and volume is adjusted with water so that the insulin concentration is 2× the required concentration. The composition is then mixed 1:1 (v/v) with a mixture of additional excipients (all at 2× the required concentration).

### Example 2

### Stability of Insulin Aspart in the Presence of Nicotinamide and Additional Excipients

The stability of insulin aspart in the formulation of currently marketed NovoRapid® rapid-acting product (formulation F1 in Table 1) was compared with that of insulin aspart in a number of nicotinamide-containing formulations (formulations F2-F17 in Table 1) following storage at 37° C. Formulation F2 contained arginine and was based on formulation K in Table 1 of WO2010/149772, which was shown to have an ultra-rapid acting pharmacodynamic/pharmacokinetic profile. The only difference between formulation F2 and formulation K of WO2010/149772 is the use of phosphate buffer instead of TRIS in order to eliminate a buffer effect in comparing with currently marketed NovoRapid®. Formulations F3-F17 were designed to study the effect on insulin aspart stability of (1) salts (2) polyols and (3) non-ionic surfactants.

TABLE 1
Compositions of formulations F1-F17 of insulin aspart tested. All formulations comprised
insulin aspart (100 U/ml), ionic zinc (0.3 mM) as ZnCl₂, phenol (16 mM) and m-cresol
(16 mM) and were adjusted to pH 7.4. Other components are listed in the table.

| | | | | | | | | | Surfactant: Polysorbate |
|---|---|---|---|---|---|---|---|---|---|
| | Sodium | Sodium | Potassium | Sodium | | | | | 20(A) or Polysorbate 80 |
| | phosphate | chloride | chloride | acetate | Arginine | Glycerol | Mannitol | Nicotinamide | (B)or Dodecyl maltoside |
| | (mM) | (mM) | (mM) | (mM) | (mM) | (mM) | (mM) | (mM) | (C) (mg/ml) |
| F1 | 7 | 10 | | | | 174 | | | |
| F2 | 7 | 10 | | | 30 | 84 | | 80 | |
| F3 | 7 | | | | 30 | 84 | | 80 | |
| F4 | 7 | | | | | 84 | | 80 | |
| F5 | 7 | | | | | 141 | | 80 | |
| F6 | 7 | | | | | | 141 | 80 | |
| F7 | 7 | 140 | | | | | | 80 | |
| F8 | 7 | 70 | | | | | | 80 | |
| F9 | 7 | 30 | | | | 83 | | 80 | |
| F10 | 7 | | 70 | | | | | 80 | |
| F11 | 7 | | | 70 | | | | 80 | |
| F12 | 7 | | | | | 141 | | 80 | 0.05 (A) |
| F13 | 7 | 70 | | | | | | 80 | 0.05 (A) |
| F14 | 7 | | | | | 141 | | 80 | 0.05 (B) |
| F15 | 7 | 70 | | | | | | 80 | 0.05 (B) |
| F16 | 7 | | | | | 141 | | 80 | 0.05 (C) |
| F17 | 7 | 70 | | | | | | 80 | 0.05 (C) |

Results of the visual assessment of formulations F1-F17 are shown in Table 2. It was surprisingly shown that the arginine-containing formulation F2 resulted in a considerably greater rate of particle formation compared with formulation F1 (i.e. formulation of NovoRapid®). Formulation F2 reached the “Fail” limit after 1 week of storage at 37° C., whilst formulation F1 only reached the limit following 3 weeks storage at the same temperature. It was also shown that removal of the 10 mM NaCl from formulation F2 had no significant impact on the rate of particle formation (F3 vs. F2). Removal of arginine from formulation F3 led to a considerable reduction in the rate of particle formation (F4 vs. F3) and it was also shown that increasing the concentration of glycerol in the arginine-free formulation (F5 vs. F4) or replacing it with mannitol, an alternative polyol, (F6 vs. F5), had only a minimal impact on the rate of particle formation. Use of salts, including sodium chloride (F7-F9), potassium chloride (F10) and sodium acetate (F11) resulted in a similar rate of particle formation to that in the presence of arginine. Only the formulation comprising the lowest concentration of sodium chloride (F7) appeared to result in a “Pass” visual score at 1 week, but reached a “Fail” score 5 at 2 weeks alongside all other formulations comprising a salt. Addition of a non-ionic surfactant to the formulations comprising either 70 mM sodium chloride (F13, F15 and F17) or 141 mM glycerol (F12, F14 and F16) resulted in a considerable reduction in the rate of particle formation. In all cases, the rate of particle formation was lower or comparable with that of formulation F1 (i.e. formulation of NovoRapid®). The formulations containing dodecyl maltoside (F16 and F17) gave the best performance.

TABLE 2
Visual scores of insulin aspart formulations F1-F17 following storage
at 37° C. Visual score 1: clear solution, virtually free of particles;
visual score 2: ~5 very small particles; visual score 3: ~10-20
very small particles; visual score 4: 20-50 particles, including larger
particles; visual score 5: >50 particles, including larger particles.

| | Visual | Visual | Visual | Visual | Visual |
|---|---|---|---|---|---|
| | score | score | score | score | score |
| | (0 weeks) | (1 week) | (2 weeks) | (3 weeks) | (4 weeks) |
| F1 | 1 | 2 | 3 | 4 | 5 |
| F2 | 1 | 4 | 5 | 5 | 5 |
| F3 | 1 | 4 | 5 | 5 | 5 |
| F4 | 1 | 3 | 3 | 4 | 4 |
| F5 | 1 | 3 | 4 | 4 | 4 |
| F6 | 1 | 3 | 3 | 4 | 4 |
| F7 | 1 | 4 | 5 | 5 | 5 |
| F8 | 1 | 4 | 5 | 5 | 5 |
| F9 | 1 | 3 | 5 | 5 | 5 |
| F10 | 1 | 4 | 5 | 5 | 5 |
| F11 | 1 | 4 | 5 | 5 | 5 |
| F12 | 1 | 1 | 2 | 3 | 4 |
| F13 | 1 | 2 | 3 | 3 | 5 |
| F14 | 1 | 2 | 3 | 4 | 4 |
| F15 | 1 | 2 | 3 | 3 | 4 |
| F16 | 1 | 1 | 1 | 2 | 2 |
| F17 | 1 | 1 | 1 | 2 | 3 |

Formation of HMWS in formulations F1-F17 is shown in Table 3 and formation of chemically related species is shown in Table 4. The arginine-containing formulation F2 resulted in a lower rate of HMWS and chemically related species compared with formulation F1 (i.e. formulation of NovoRapid®). Removal of arginine from formulation F3 led to an impairment of stability, both with respect to HMWS and with respect to chemically related species (F4 vs. F3). Increasing the concentration of glycerol in the arginine-free formulation (F5 vs. F4) or replacing it with mannitol, an alternative polyol, (F6 vs. F5), had only a minimal impact on the stability. Use of salts, including sodium chloride (F7-F9), potassium chloride (F10) and sodium acetate (F11) resulted in better stability, both with respect to HMWS and with respect to chemically related species compared with formulations that did not contain salts. The beneficial effect of a salt appeared to be concentration-dependent (F7-F9), and in all cases, it was better than that of the formulation F1 (i.e. formulation of NovoRapid®). Addition of a non-ionic surfactant to the formulations comprising either 70 mM sodium chloride (F13, F15 and F17) or 141 mM glycerol (F12, F14 and F16) resulted in only minimal impact of stability both with respect to HMWS and with respect to chemically related species

Overall, only formulations comprising a non-ionic surfactant and a salt resulted in stability that was considerably better in all aspects than that achieved in the marketed formulation of NovoRapid®.

TABLE 3
Increase in HMWS (vs. start) in insulin aspart formulations
F1-F17 assessed by SEC following storage at 37° C.

| | Δ % HMWS (2 weeks vs. start) | Δ % HMWS (4 weeks vs. start) |
|---|---|---|
| F1 | 0.39 | 0.69 |
| F2 | 0.20 | 0.37 |
| F3 | 0.19 | 0.35 |
| F4 | 0.55 | 1.01 |
| F5 | 0.53 | 0.96 |
| F6 | 0.43 | 0.91 |
| F7 | 0.21 | 0.41 |
| F8 | 0.25 | 0.52 |
| F9 | 0.33 | 0.63 |
| F10 | 0.25 | 0.66 |
| F11 | 0.29 | 0.70 |
| F12 | 0.60 | 1.20 |
| F13 | 0.28 | 0.58 |
| F14 | 0.60 | 1.21 |
| F15 | 0.30 | 0.55 |
| F16 | 0.66 | 1.23 |
| F17 | 0.26 | 0.52 |

TABLE 4
Increase in chemically related species insulin (vs.
start) aspart formulations F1-F17 assessed by reversed-
phase chromatography following storage at 37° C.

| | Δ % chemically related species | Δ % chemically related species |
|---|---|---|
| | (2 weeks vs. start) | (4 weeks vs. start) |
| F1 | 1.56 | 3.35 |
| F2 | 0.98 | 2.09 |
| F3 | 1.00 | 2.14 |
| F4 | 1.49 | 3.39 |
| F5 | 1.52 | 3.38 |
| F6 | 1.39 | 2.99 |
| F7 | 0.82 | 1.64 |
| F8 | 0.98 | 1.84 |
| F9 | 1.22 | 2.59 |
| F10 | 0.86 | 1.75 |
| F11 | 0.97 | 2.16 |
| F12 | 1.71 | 3.37 |
| F13 | 1.00 | 1.89 |
| F14 | 1.6 | 3.33 |
| F15 | 1.02 | 1.80 |
| F16 | 1.72 | 3.34 |
| F17 | 0.95 | 1.68 |

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

All publications, patents, patent applications, internet sites, and accession numbers/database sequences (including both polynucleotide and polypeptide sequences) cited are herein incorporated by reference in their entirety for all purposes to the same extent as if each individual publication, patent, patent application, internet site, or accession number/database sequence were specifically and individually indicated to be so incorporated by reference.

SEQUENCE LISTING:

SEQ ID NO: 1: GIVEQCCTSICSLYQLENYCN
SEQ ID NO: 2: FVNQHLCGSHLVEALYLVCGERGFFYTPKT
SEQ ID NO: 3: FVNQHLCGSHLVEALYLVCGERGFFYTKPT
SEQ ID NO: 4: FVNQHLCGSHLVEALYLVCGERGFFYTDKT
SEQ ID NO: 5: FVKQHLCGSHLVEALYLVCGERGFFYTPET

1. An aqueous liquid pharmaceutical formulation comprising (i) an insulin compound, (ii) ionic zinc, (iii) a nicotinic compound (iv) a non-ionic surfactant; and (v) a salt selected from the salts formed between Group 1 metals and a mono or divalent anion.

2. The formulation according to claim 1
wherein the insulin compound is insulin lispro;
wherein the insulin compound is insulin aspart;
wherein the insulin compound is insulin glulisine; or
wherein the insulin compound is recombinant human insulin.

3.-5. (canceled)

6. The formulation according to claim 1, wherein the insulin compound is present at a concentration of 10-1000 U/ml.

7. The formulation according to claim 1, wherein the nicotinic compound is nicotinamide.

8. The formulation according to claim 1, wherein the nicotinic compound is nicotinic acid or a salt thereof.

9. The formulation according to claim 1, wherein the nicotinic compound is present at a concentration of 10-150 mM.

10. The formulation according to claim 1 wherein the non-ionic surfactant is an alkyl glycoside.

11. The formulation according to claim 10 wherein the alkyl glycoside is dodecyl maltoside.

12. The formulation according to claim 1 wherein the non-ionic surfactant is a polysorbate surfactant which is polysorbate 20 or polysorbate 80.

13. (canceled)

14. The formulation according to claim 1 wherein the non-ionic surfactant is an alkyl ether of polyethylene glycol which is selected from polyethylene glycol (2) dodecyl ether, polyethylene glycol (2) oleyl ether and polyethylene glycol (2) hexadecyl ether.

15. (canceled)

16. The formulation according to claim 1 wherein the non-ionic surfactant is a block copolymer of polyethylene glycol and polypropylene glycol which is poloxamer 188, poloxamer 407, poloxamer 171 or poloxamer 185.

17. (canceled)

18. The formulation according to claim 1 wherein the non-ionic surfactant is an alkylphenyl ether of polyethylene glycol which is 4-(1,1,3,3-tetramethylbutyl)phenyl-polyethylene glycol.

19. (canceled)

20. The formulation according to claim 1 wherein the surfactant is present at a concentration of 1-1000 μg/ml.

21. The formulation according to claim 20 wherein the surfactant is present at a concentration of 10-100 μg/ml.

22. The formulation according to claim 1 wherein the salt selected from the salts formed between Group 1 metals and a mono or divalent anion is a sodium salt of a mono or divalent anion.

23. The formulation according to claim 1 wherein the anion is a monovalent anion.

24. The formulation according to claim 1 wherein the anion is an inorganic anion.

25. The formulation according to claim 1 wherein the anion is an organic anion.

26. The formulation according to claim 24 wherein the anion is chloride.

27. The formulation according to claim 25 wherein the anion is acetate.

28. The formulation according to claim 1 wherein the salt selected from the salts formed between Group 1 metals and mono or divalent anions is present in the formulation at a concentration of 30-200 mM

29. The formulation according to claim 1, wherein ionic zinc is present in the formulation at a concentration of 0.05% or more by weight of zinc based on the weight of insulin compound in the formulation; or
wherein the ionic zinc is present at a concentration of 0.5-1% by weight of zinc based on the weight of insulin compound in the formulation.

30. (canceled)

31. The formulation according to claim 1 comprising an uncharged tonicity modifying agent selected from the group consisting of trehalose, mannitol, glycerol, or 1,2-propanediol.

32. (canceled)

33. The formulation according to claim 31, wherein the uncharged tonicity modifying agent is glycerol.

34. The formulation according to claim 1, wherein the formulation is isotonic.

35. The formulation according to claim 1, wherein the pH is in the range 5.5 to 9.0.

36. The formulation according to claim 1, comprising a preservative selected from the group consisting of phenol, m-cresol, chlorocresol, benzyl alcohol, propylparaben, methylparaben, benzalkonium chloride, and benzethonium chloride.

37. (canceled)

38. The formulation according to claim 1 comprising zinc binding species selected from species having a log K with respect to zinc ion binding of 4.5 or more at 25° C.

39. The formulation according to claim 1 which is substantially free of zinc binding species selected from species having a log K with respect to zinc ion binding of 4.5 or more at 25° C.

40. (canceled)

41. A method of treatment of diabetes mellitus which comprises administering to a subject in need thereof an effective amount of a formulation according to claim 1.

42. A container containing one dose or a plurality of doses of the formulation according to claim 1.

43. An injection device for single or multiple use comprising a container containing one dose or a plurality of doses of the formulation according to claim 1 together with an injection needle.

44. A medical device comprising a reservoir comprising plurality of doses of the formulation according to claim 1 and a pump adapted for automatic or remote operation such that upon automatic or remote operation one or more doses of the formulation is administered to the body.

45. A dry solid pharmaceutical composition suitable for reconstitution with an aqueous medium which comprises (i) an insulin compound, (ii) ionic zinc, (iii) a nicotinic compound (iv) a non-ionic surfactant; and (v) a salt selected from the salts formed between Group 1 metals and a mono or divalent anion.

46. A method of preparing a formulation according to claim 1 which comprises dissolving a dry solid pharmaceutical composition suitable for reconstitution with an aqueous medium which comprises (i) an insulin compound, (ii) ionic zinc, (iii) a nicotinic compound (iv) a non-ionic surfactant; and (v) a salt selected from the salts formed between Group 1 metals and a mono or divalent anion, in an aqueous medium.

47. A method of improving the storage stability of an aqueous liquid pharmaceutical formulation comprising (i) an insulin compound, (ii) ionic zinc, (iii) a nicotinic compound and (iv) a salt selected from the salts formed between Group 1 metals and a mono or divalent anion which comprises adding a non-ionic surfactant to the formulation.

48. (canceled)

49. A method of improving the storage stability of an aqueous liquid pharmaceutical formulation comprising (i) an insulin compound, (ii) ionic zinc and (iii) a nicotinic compound which comprises adding a non-ionic surfactant and a salt selected from the salts formed between Group 1 metals and a mono or divalent anion to the formulation.

50. (canceled)
